(19) United States
(12) Patent Application Publication
(10) Pub. No.: US 2018/0177964 A1
(43) Pub. Date: Jun. 28, 2018

(54) GLOTTIS MASK AIRWAY

(71) Applicant: Tianjin Medan Medical Corp., Tianjin (CN)

(72) Inventor: Hui Zhu, Tianjin (CN)

(73) Assignee: Tianjin Medan Medical Corp., Tianjin (CN)

(21) Appl. No.: 15/851,726

(22) Filed: Dec. 21, 2017

(30) Foreign Application Priority Data
Dec. 22, 2016 (CN) 201611199621.2
Dec. 22, 2016 (CN) 201611201125.6

(51) Int. Cl. A61M 16/04

(52) U.S. Cl. CPC A61M 16/0447; A61M 16/0443; A61M 2210/1028; A61M 16/0415; A61M 16/0431; A61M 16/0463

(57) ABSTRACT

Glottis Mask Airway (GMA)—an aperture of larynx sealed airway for human ventilation includes a main airway; a non-inflatable cuff at the distal end of the main airway; the shape of the cuff is matched the shape of aperture of larynx perfectly. There is a sealing ring in the device to seal the intersection angle between the aperture of larynx and the wall of pharynx to prevent leakage during the ventilation. There is an epiglottis holder within the cuff where corresponding the epiglottis to prevent the epiglottis falls into the main airway cause the airway block. Plus there are 1) C-channel for gastric tube insertion; 2) at the proximal end of the main airway an openside airway is able to push ETT to the proper position at once; 3) a tongue base rest as the device stabilizer to stabilize the device in using.

### CROSS-REFERENCE TO RELATED APPLICATION

This application claims the priority benefits of China application serial no. 201611201125.6, filed on Dec. 22, 2016, and China application serial no. 201611199621.2, filed on Dec. 22, 2016. The entirety of each of the above-mentioned patent applications is hereby incorporated by reference herein and made a part of specification.

### BACKGROUND OF THE INVENTION

### 1. Technical Field

The present invention belongs to the field of medical devices, and more specifically, an aperture of larynx sealed ventilion device.

### 2. Description of Related Technology

A Laryngeal Mask Airway (LMA) i.e Supraglottic Airway (SGA) is used in clinical ventilation; emergency ventilation and intensive care unit breathe support. Laryngeal Mask Airway can be classified as inflatable LMA and non-inflatable LMA. Non-inflatable LMA can be classified as rigid material and soft material LMA.

The weakness of the present LMA

1. Inflatable LMA: a), operation is complex; b), the inflatable cuff is not easy to handle: lower inflation causing leakage; over inflation causing compression trauma to the surrounding tissue and structure.

2. Non-inflatable rigid material LMA: a) the material is rigid; it will hurt the isthmus of fauces when it goes through it; b) as well as hurt the soft tissue surround the pharynx and larynx when the device is placed inside the mouth cavity.

3. Non-inflatable soft tissue LMA: present device a), form seal by anatomical fit to the laryngeal framework, there will be a leakage. b), Epiglottis rest is a misposition where the epiglottis won't rest the site.

4. In a part of LMA (both inflatable and non-inflatable) there is a function which is ETT inserted through the device. In the cases the ETT can't be placed into the right position in one time; it has to use a pushing rod to push the ETT to the proper position so as to keep the ETT and take out the device.

5. In a part of LMA (both inflatable and non-inflatable) there is a) channel to insert gastric tube but there are two weaknesses of the gastric channel: a), the distal end of the gastric channel is on the tip of the device which the gastric tube will be blocked by main tracheal. b), gastric channel is a closed channel, the gastric tube has to be taken out together with the device when you take out the device.

6. The stability of the present LMA is a question after it's placement.

### SUMMARY OF THE INVENTION

The present invention provides a glottis mask airway as claimed in the claims.

The present invention is designed base on the anatomy specification of the aperture of larynx, the anatomy location of aperture of larynx and the anatomy structure around the aperture of larynx.

The anatomy specification of aperture of larynx: it consists of epiglottis, aryepiglottic folds and inter arytenoid notch. They are same in shape, same location, and no evolution.

The anatomy location of aperture of larynx: the location is fixed and surrounded by soft tissue.

The anatomy structure of aperture of larynx: the wall of pharynx over the aperture of larynx; there is an intersection angle between the pharynx wall and the aperture of larynx.

The glottis mask airway, which is placed over the aperture of larynx by a bowl shape cuff, the distal opening of the device's airway toward the glottis directly, and the device is named as Glottis Mask Airway or GMA for short.

A glottis mask airway (GMA) includes an arc main airway and a non-inflatable, gel-like and bowl-shaped cuff which is rest on a cuff holder which is at the distal end of the main airway; the shape of the cuff matches the shape of anatomy structure of the aperture of larynx perfectly; the cuff above over the aperture of larynx; there is a sealing ring surrounding the edge of the cuff which seals the intersection angle between aperture of larynx and the wall of pharynx.

Preferably, the sealing ring is on the top of the inner edge of the cuff; it will fill into the intersection angle between the aperture of larynx and the wall of pharynx.

In case the epiglottis falls into the airway it will fold toward to the glottis caused by air flow, the epiglottis will block the airway. Preferably, there is an epiglottis holder within the cuff where corresponds the epiglottis, the epiglottis rests on the epiglottis holder. The epiglottis remains in open all the time. The epiglottis holder will prevent the epiglottis falls into the airway. That is, the epiglottis is supported by the epiglottis holder so as to prevent the epiglottis folds towards a glottis by air flow to block the main airway.

Preferably, there is a C-channel (for gastric tube insertion) on the one side of the main airway goes along from the proximal end down to the back of cuff holder. C-channel is for the gastric tube insertion; C-Channel is able to remain the gastric tube in place while the GMA is removed.

Preferably, the distal end of C-channel at the back of the cuff holder, facing direct to the open of the esophagus.

Preferably, there is tongue base rest which is a horizontal plane to hold the tongue base; the plane extending to both sides but not over the horizontal wide of the cuff; it's located at the proximal side of cuff holder corresponding to the tongue base; tongue base rests on the plane it's a stabilizer of the device.

Preferably, there is an open side which starts at the proximal end of the device; takes around ¼ of the length of the whole device on the other side of the main airway. The function of the open side to push the ETT to the proper position when you have to insert ETT through the device.

Preferably, the open side is detached or locked by a locker device.

Preferably, there is a dovetail groove on each of sides of the open edges separately, and a dovetail tenon is provided on each of two sides of the locker device corresponding to the dovetail groove separately.

Preferably, the locker device is integrated with the connecter which connects to anesthesia machine and the bite blocker.

Preferably, the cross section of the main airway is square, four corners of the airway with a smooth transition.

### BRIEF DESCRIPTION OF THE SEVERAL VIEWS OF THE DRAWINGS

In the figures:

FIG. 1 is a schematic structural view of Embodiment 1 of the present invention.

FIG. 2 is a schematic view of the back of Embodiment 1 of the present invention.

FIG. 3 is a schematic structural view of Embodiment 2 of the present invention.

FIG. 4 is a schematic view of the separated open edge of the GMA in Embodiment 2 of the present invention.

FIG. 5 is a schematic view of the locking mechanism in Embodiment 2 of the present invention.

FIG. 6 is a schematic view of the contrasted position between the human larynx and the present invention.

FIG. 7 is a schematic view of the intersection angel between the aperture of larynx and pharynx wall.

### DETAILED DESCRIPTION OF THE INVENTION

The present invention on the basis of study anatomy structure of pharynx, larynx from literature; standard text book; cadaveric model as well as clinical study. After the study we found the features of aperture of larynx:

(1) The location is fix and no evolution; the opening of larynx; under the wall of pharynx.

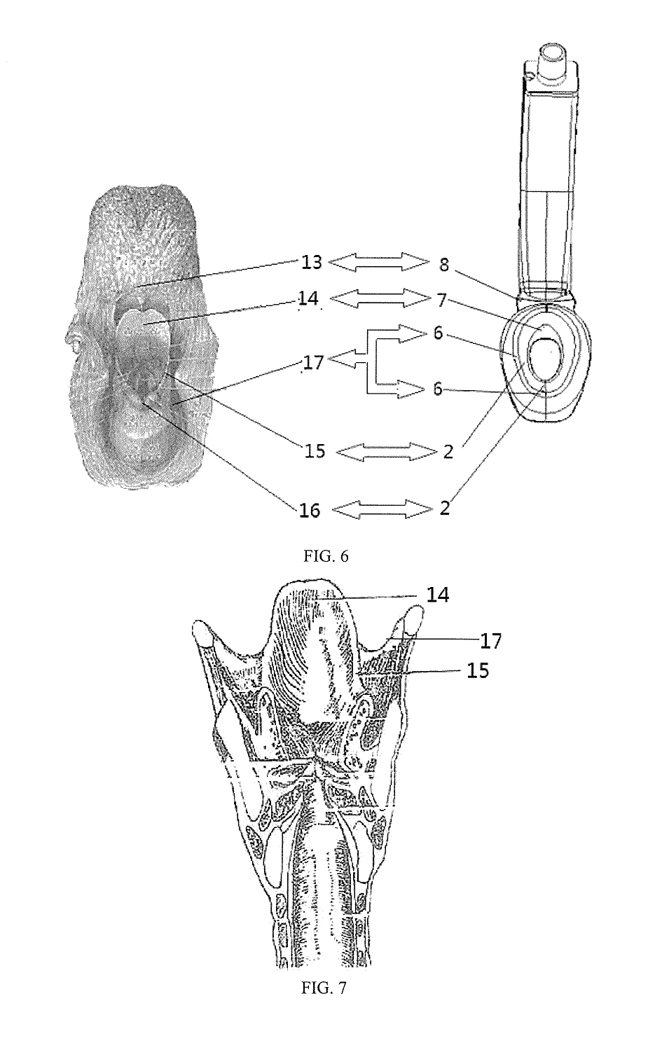

(2) The structure is fixed no evolution (as shown in FIG. 6, including the epiglottis 14, the aryepiglottic fold 15; inter arytenoid notch 16).

(3) The shape is fixed surrounded with the soft tissue.

(4). There is an intersection angle between the aperture of larynx and pharynx wall 17.

On the basis of the features of aperture of larynx the present invention designed a cuff 2 which is match the shape of aperture of larynx; design a sealing ring 6 on the top of the inner edge of the cuff 2; the sealing ring 6 goes into the intersection angel between the aperture of larynx and pharynx wall 17 as well as sealed the intersection angle. The cuff 2 of the device is made of a soft material which is as soft as the larynx and pharynx tissue. It's a bowl shape, no inflatable, gel like cuff. The present invention above over the aperture of larynx and the distal open of the airway toward the glottis directly, named it the glottis mask airway (GMA) for short.

The following two embodiments are given for the specific application of the present invention.

### Embodiment 1 (Without Open Side)

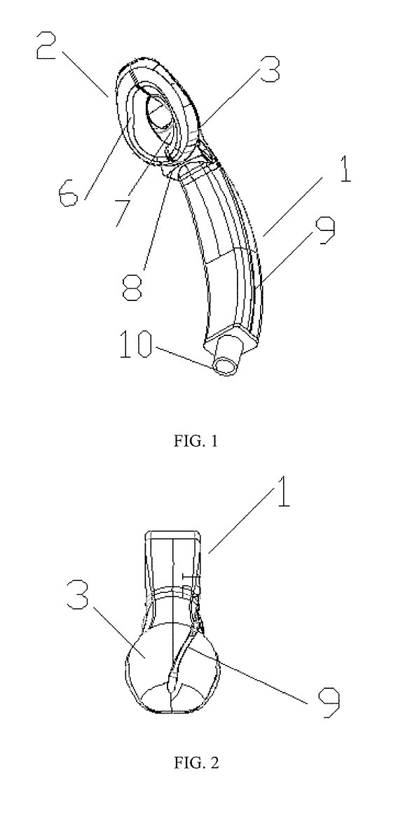

As shown in FIGS. 1 and 2, the present invention provides a glottis mask airway which includes a main airway 1 and a non-inflatable, gel-like and bowl-shaped cuff 2 which is rest on a cuff holder 3 which is at the distal end of the main airway 1. The shape of the cuff 2 matches the shape of anatomy structure of the aperture of larynx perfectly. The cuff 2 is above over the aperture of larynx.

FIG. 6 is a schematic view of the anatomy landmark correspond to the cuff structure. As shown in FIG. 6, the shape of the cuff 2 matches the shape of anatomy structure of the aperture of larynx perfectly, and the cuff 2 is above over the aperture of larynx.

The present invention designs a sealing ring 6 on the top of the inner edge of the cuff 2, and the sealing ring 6 goes into the intersection angel between the aperture of larynx and pharynx wall 17 as well as sealed the intersection angle. FIG. 7 is a schematic view of the intersection angel between the aperture of larynx and pharynx wall 17.

The present invention: there is an epiglottis holder 7 within the cuff 2 where corresponding the epiglottis. The epiglottis rests on it, keeps the glottis open, prevent the glottis from falling down to the airway by air flow cause the airway block.

The present invention: a C-channel 9 (for gastric tube insertion) which is on the one side of the main airway 1 goes along from the proximal end down to the back of cuff holder 3. The C-channel 9 is for the gastric tube insertion. The C-Channel 9 is able to remain the gastric tube in place while the GMA is removed. The distal end of C-channel 9 is at the back of the cuff holder 3, faces direct to the open of the esophagus. The distal end of C-channel 9 at the back of the cuff holder 3 is able to prevent the gastric tube from being blocked by main tracheal when insert the gastric tube.

The present invention: there is a tongue base rest 8 which is a horizontal plane to hold a tongue base 13. The plane extending to both sides but not over the horizontal wide of the cuff, and it's located at the proximal side of cuff holder 3 corresponding to the tongue base. The tongue base 13 rests on the plane. The tongue base rest 8 is the stabilizer of the device.

The present invention: the cross section of the main airway 1 is square, four corners of the airway with a smooth transition.

The following problems are solved within Embodiment 1: (1) the leakage of the airway; (2) the post operation throat pain and other complications caused by the compression of the surrounding tissue, nerves caused by the inflatable cuff; (3) the hurt of the isthmus of fauces caused by the rigid material LMA; (4) the risk of the airway block caused by the “epiglottis falls into the airway” during the ventilation; (5) the stability of the device in use; (6) the problem of keep the gastric tube when take out the device; the present invention has improved/solved the problems above completely or partially, provide an easy to use; safety; and no hurt to patient; non-inflatable Laryngeal Mask Airway.

### Embodiment 2 (With Side Open)

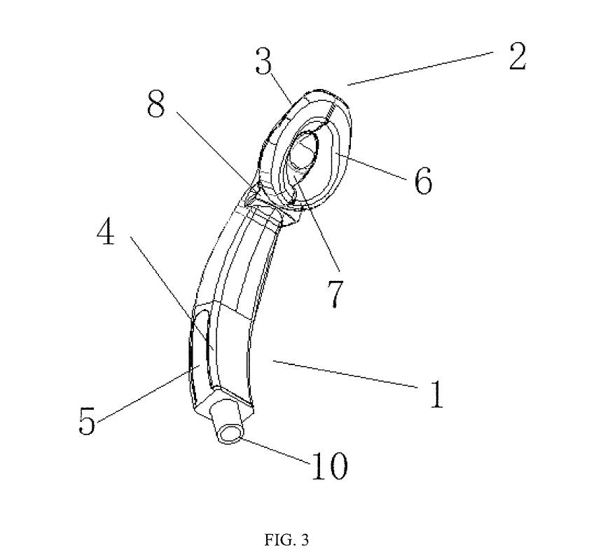

As shown in FIGS. 3, 4 and 5, the present invention provides a glottis mask airway which includes a main airway 1 and a non-inflatable, gel-like and bowl-shaped cuff 2 which is rest on a cuff holder 3 which is at the distal end of the main airway 1. The shape of the cuff 2 matches the shape of anatomy structure of the aperture of larynx perfectly. The cuff 2 above over the aperture of larynx. There is an open side 4 at the ⅓-¼ proximal end of the main airway 1.

FIG. 6 is a schematic view of the anatomy landmark correspond to the cuff structure. As shown in FIG. 6, the shape of the cuff 2 matches the shape of anatomy structure of the aperture of larynx perfectly, and the cuff 2 is above over the aperture of larynx.

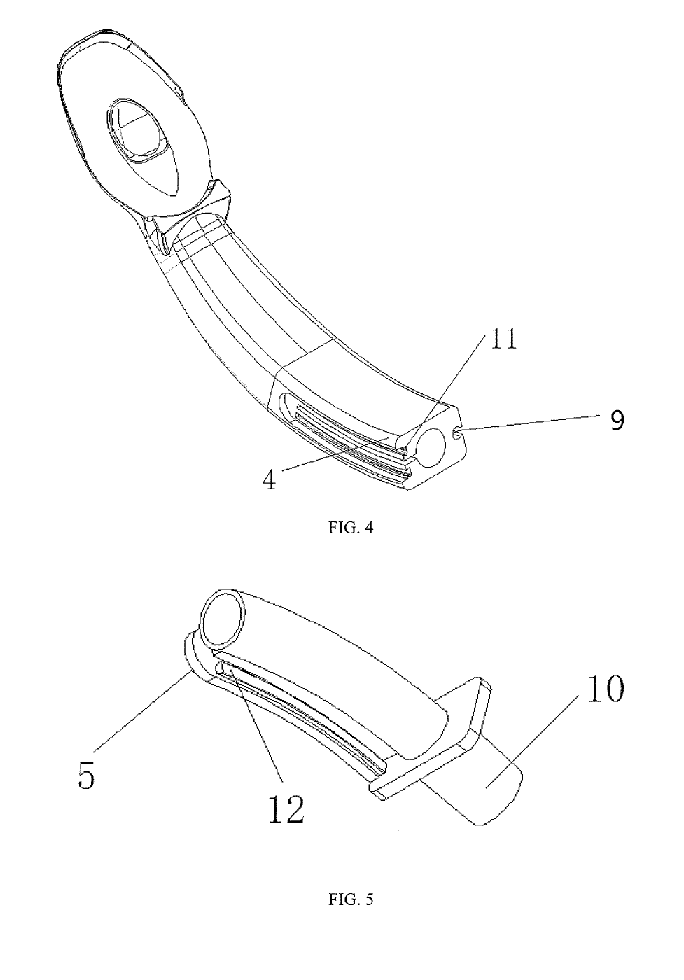

There is an open side 4 (shown in FIG. 4) which starts at the proximal end of the device takes ¼-⅓ of the length of the whole device on the other side other side of the maim airway 1. The function of the open side 4 to push the ETT to the proper position when you have to insert ETT through the device.

The open side 4 is detached or locked by a locker device 5.

There is a dovetail groove 11 on each of sides of the open edges separately, and a dovetail tenon 12 is provided on each of two sides of the locker device corresponding to the dovetail groove 11 separately.

The locker device is integrated with the connecter which connects to anesthesia machine and the bite blocker 10.

Except side open Embodiment 1 is exact same to Embodiment 2

The present invention designed a sealing ring 6 on the top of the inner edge of the cuff 2; the sealing ring 6 goes into the intersection angel between the aperture of larynx and pharynx wall 17 as well as sealed the intersection angle. FIG. 7 is a schematic view of the intersection angel between the aperture of larynx and pharynx wall.

The present invention: there is an epiglottis holder 7 within the cuff 2 where corresponds the epiglottis. The epiglottis rests on it, keeps the glottis open; prevent the glottis falls down to the airway by air flow cause the airway block.

The present invention: there is a C-channel 9 (for gastric tube insertion) on the one side of the main airway 1 goes along from the proximal end down to the back of cuff holder 3. The C-channel 9 is for the gastric tube insertion. The C-Channel 9 is able to remain the gastric tube in place while the GMA is removed. The distal end of the C-channel 9 at the back of the cuff holder 3, facing direct to the open of the esophagus. The distal end of the C-channel 9 at the back of the cuff holder 3 is able to prevent the gastric tube from being blocked by main tracheal when insert the gastric tube.

The present invention: there is a tongue base rest 8 which a horizontal plane to hold the tongue base. The plane extending to both sides but not over the horizontal wide of the cuff, it's located at the proximal side of cuff holder 3 corresponding to the tongue base; tongue base 13 rest on the plane, and it's the stabilizer of the device.

The present invention: the cross section of the main airway 1 is square, four corners of the airway have smooth transitions.

The following problems are solved within Embodiment 1: (1) the leakage of the airway; (2) the post operation throat pain and other complications caused by the compression of the surrounding tissue nerves caused by the inflatable cuff; (3) the hurt of the isthmus of fauces caused by the rigid material LMA; (4) the risk of the airway block caused by the “epiglottis falls into the airway” during the ventilation; (5) the stability of the device in use; (6) the problem of keep the gastric tube when take out the device; (7) insert ETT through the device has to use a pushing rod to push the ETT to the proper position. The present invention has improved/solved the problems above completely or partially, provide an easy to use; safety; and no hurt to patient; non-inflatable Laryngeal Mask Airway.

The basic principle, main characteristics, implementation and other information of the present invention have been described above. However, the present invention is not limited to the foregoing process of implementation; various changes and modifications may be made to the present invention without departing from the spirit and scope of the invention. Therefore, any changes and modifications without departing from the scope of the present invention shall be construed as being included therein.

What is claimed is:

1. A glottis mask airway (GMA), comprising:
a main airway; and
a non-inflatable, gel-like and bowl-shaped cuff which is rest on a cuff holder which is at a distal end of the main airway, wherein a shape of the cuff matches a shape of an anatomy structure of an aperture of a larynx, the cuff is above over the aperture of the larynx, a sealing ring surrounds an inner edge of the cuff which seals an intersection angle between the aperture of the larynx and a wall of a pharynx.

2. The GMA as claimed in claim 1, wherein the sealing ring is on a top of the inner edge of the cuff, the sealing ring fills into the intersection angle between the aperture of the larynx and the wall of the pharynx.

3. The GMA as claimed in claim 1, wherein an epiglottis holder is disposed within the cuff where correspond an epiglottis, the epiglottis rests on the epiglottis holder; the epiglottis remains in open all the time, the epiglottis is supported by the epiglottis holder so as to prevent the epiglottis folds towards a glottis by air flow to block the main airway.

4. The GMA as claimed in claim 1, wherein there is a C-channel formed on one side of the main airway goes along from a proximal end down to a back of the cuff holder, the C-channel is for a gastric tube insertion, and the C-channel is able to remain the gastric tube in place while the GMA is removed.

5. The GMA as claimed in claim 4, wherein a distal end of the C-channel at the back of the cuff holder, directly faces to an open of an esophagus.

6. The GMA as claimed in claim 1, wherein a tongue base rest which is a horizontal plane to hold a tongue base; the horizontal plane extends to both sides but not over a horizontal wide of the cuff, the tongue base rest is located at a proximal side of the cuff holder corresponding to the tongue base, and the tongue base rests on the plane, and the tongue base rest is a stabilizer of the glottis mask airway.

7. The GMA as claimed in claim 1, wherein an open side which starts at a proximal end of the main airway takes around ¼ of a length of the whole main airway on a side of the arc maim airway, the function of the open side is to push an endotracheal tube (ETT) to a position when you have to insert the ETT through the main airway.

8. The GMA as claimed in claim 7, wherein the open side is detached or locked by a locker device.

9. The GMA as claimed in claim 8, wherein a dovetail groove on each of sides of the open edges separately, and a dovetail tenon is provided on each of two sides of the locker device corresponding to the dovetail groove separately.

10. The GMA as claimed in claim 8, wherein the locker device is integrated with a connecter which connects to an anesthesia machine and a bite blocker.

11. The GMA as claimed in claim 9, wherein the locker device is integrated with a connecter which connects to an anesthesia machine and a bite blocker.

12. The GMA as claimed in claim 1, wherein a cross section of the main airway is square, and four corners of the square have smooth transitions.
